(19) United States
(12) Patent Application Publication
(10) Pub. No.: US 2024/0165148 A1
(43) Pub. Date: May 23, 2024

(54) HYALURONIC ACID-CONJUGATED DIPALMITOYL PHOSPHATIDYL ETHANOLAMINE IN COMBINATION WITH NON-STEROIDAL ANTI-INFLAMMATORY DRUGS (NSAIDS) FOR TREATING OR ALLEVIATING INFLAMMATORY DISEASES

(71) Applicant: Saul YEDGAR, Jerusalem (IL)

(72) Inventor: Saul YEDGAR, Jerusalem (IL)

(21) Appl. No.: 18/549,042

(22) PCT Filed: Mar. 13, 2022
(86) PCT No.: PCT/IL2022/050282

§ 371 Date: Sep. 5, 2023

Related U.S. Application Data
(60) Provisional application No. 63/160,947, filed on Mar. 15, 2021.

(51) Int. Cl. A61K 31/728; A61K 31/365; A61K 31/415; A61K 31/612; A61K 47/24; A61P 29/00

(52) U.S. Cl. CPC A61K 31/728; A61K 31/365; A61K 31/415; A61K 31/612; A61K 47/24; A61P 29/00

(57) ABSTRACT

The present invention relates to combination therapies and compositions comprising a lipid conjugate composed of dipalmitoyl- phosphatidyl-ethanol-amine (DPPE) and Hyaluronic Acid (Hy) in combination with a non-steroidal anti-inflammatory drug (NSAID), and uses of same in treating inflammatory and/or allergic diseases or conditions.

### CROSS REFERENCE TO RELATED APPLICATIONS

This application claims the benefit of United States Provisional Patent Application Ser. Number 63/160,947, filed Mar. 15, 2021, hereby fully incorporated by reference herein.

### FIELD OF THE PRESENT INVENTION

The present invention generally pertains to compositions comprising a combination of a lipid conjugate, denoted HyDPPE, composed of dipalmitoyl-phosphatidyl-ethanol-amine (DPPE) conjugated with Hyaluronic Acid (Hy) and non-steroidal anti-inflammatory drugs (NSAID), in particular cyclooxygenase-2 inhibitors, and their uses in inflammatory disease, in particular in treating or alleviating inflammatory and/or allergic diseases.

### BACKGROUND OF THE INVENTION

Commonly used nonsteroidal anti-inflammatory drugs (NSAIDs), primarily cyclooxygenase (COX) inhibitors, and steroids have been employed for the treatment of inflammatory conditions and related symptoms.

The initiation of inflammatory/allergic processes involves two key activities: First, degradation of cell membrane lipids by the “inflammatory enzyme” secretory phospholipase A2 (sPLA2), leading to the cascade of inflammatory lipid mediators (ILM), produced by the COX pathways, e.g., prostaglandins and thromboxanes and the lipoxygenase (LO) pathways, e.g., leukotrienes. Second Activity is degradation of the cell-surface glycosaminoglycans (GAG), which protect cells and tissues from damaging agents, such as free radicals, endotoxins, and enzymes that promote cancer metastasis.

We have found a useful compound, which consists of a PLA2 inhibiting lipid, specifically dipalmitoyl-phosphatidyl-ethanol-amine (DPPE), which when conjugated to Hyaluronic Acid (HA Hy) (the conjugate referred to as HyDPPE) can promote modulation of ILM overproduction, bringing levels back to normal, basal levels following inflammatory incitement (in contrast to the selective inhibition by COX inhibitors, e.g. Vioxx® which are associated with severe side effects). Moreover, use of these compounds can enrich the cell surface protective GAG layer, providing added benefit.

As conjugates HyDPPE has shown excellent safety and found effective in pre-clinical, e.g., animal models of Asthma, IBD, Sepsis, CNS inflammation EAE, Conjunctivitis, Lung metastasis, Atherosclerosis; and clinical studies, e.g., dermatitis, allergic rhinitis, ex vivo chronic rhinosinusitis, using diverse methods of administration, it would seem that this class of conjugates as a whole, can be effectively applied to the treatment of numerous diseases of inflammatory etiology. Yet there remains a need, given the great demand to identify additional anti-inflammatory drugs that outperform the existing therapies to date.

### SUMMARY OF THE INVENTION

Surprisingly, it has now been demonstrated herein that combining NSAIDs/COX2 inhibitors specifically with HyDPPE provides superior results, even though they target a common pathway.

This invention therefore provides, in some embodiments, for the combination therapy of a therapeutically effective amount of an NSAID and HY-DPPE and compositions comprising the same and joint or staggered treatment of a subject with same and uses thereof.

The invention therefore provides, in some embodiments, for the combination therapy of a therapeutically effective amount of a COX inhibitor and HyDPPE and compositions comprising the same and joint or staggered treatment of a subject with same and uses thereof.

The invention therefore provides, in some embodiments, for the combination therapy of a therapeutically effective amount of an NSAID, and in some embodiments, specifically aCOX2 inhibitor and HyDPPE and compositions comprising the same and joint or staggered treatment of a subject with same and uses thereof.

DESCRIPTION OF THE DRAWINGS

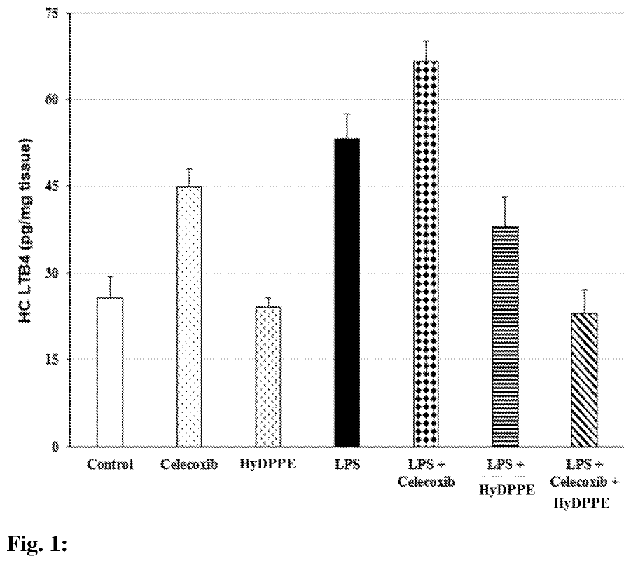

FIG. 1: Effect of Celecoxib (COX-2 inhibitor) and/or HyDPPE on LTB4 production in the hippocampus (HC) of LPS-stimulated rats.

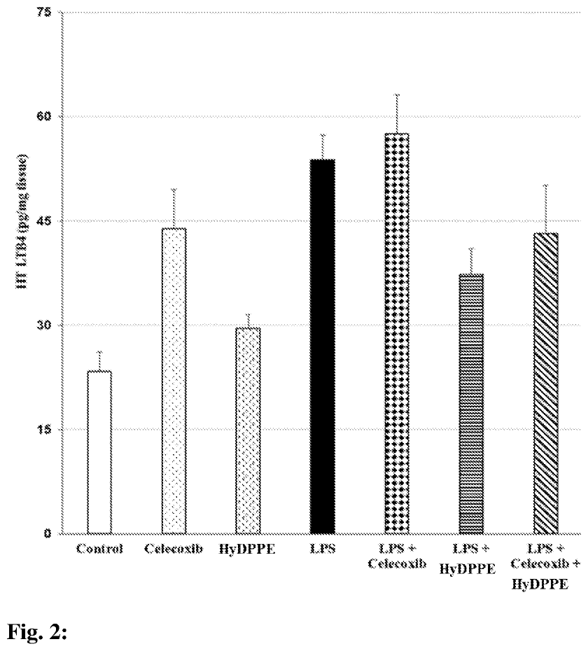

FIG. 2: Effect of Celecoxib and/or MFAID on LTB4 production in the Hyppothalamus (HT) of LPS-stimulated rats:

### DETAILED DESCRIPTION OF THE INVENTION

This invention addresses a long-felt need for optimizing treatments of inflammatory and/or allergic diseases and/or conditions, in finding a uniquely effective combination therapy of a therapeutically effective amount of a non-steroidal anti-inflammatory drug (NSAID) and a therapeutically effective amount of conjugate of di-palmitoyl (C-16) phosphatidyl ethanolamine and hyaluronic acid (HyDPPE).

As described herein, surprisingly, while HyDPPE administration alone reversed the increased LTB4 production seen induced individually by LPS and COX-2 inhibitors in hippocampus (HC) samples, the combination of HyDPPE and the COX-2 inhibitor Celecoxib showed a highly significant reduction in LTB4, indicating the unexpected, superior activity of the combination therapy in early inflammation/allergic pathogenesis.

Accordingly, this invention provides a combination therapy for treating an inflammatory or allergic disease or condition, said combination therapy comprising a therapeutically effective amount of a non-steroidal anti-inflammatory drug (NSAID) and a therapeutically effective amount of conjugate of di-palmitoyl (C-16) phosphatidyl ethanolamine and hyaluronic acid (HyDPPE).

Without being bound by theory, in some aspects, the invention is directed to the potential application of HyDPPE being particularly effective when administered in combination with one or more NSAIDs (e.g. COXIB), availing the opportunity to on the one hand, harness the utility of the NSAID, while concurrently preventing its adverse effects (by reducing arachidonic acid (AA) production and subsequent reduction of pathogenic eicosanoids, such as thromboxane (TX) or leukotrienes (LTs).

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

HyDPPE, as referred to herein may be characterized by a structure of Formula I, as follows:

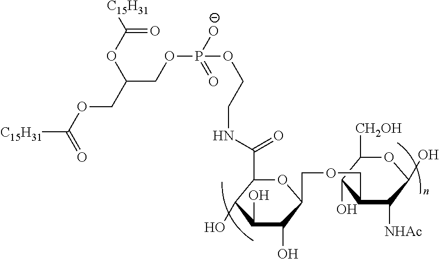

Where n is an integer ranging from 1-1000, or as is commonly found in natural sources of hyaluronic acids. In some embodiments, n ranges from 1-500, or in some embodiments, n ranges from 1-400, or in some embodiments, n ranges from 1-300, or in some embodiments, n ranges from 1-200, or in some embodiments, n ranges from 1-100, or in some embodiments, n ranges from 1-50, or in some embodiments, n ranges from 1-40, or in some embodiments, 1-30, or in some embodiments, 1-25, or in some embodiments, 1-20, or in some embodiments, 1-15, or in some embodiments, 1-10, or in some embodiments, any number of repeating units in subranges of the listed ranges herein.

In some embodiments, the hyaluronic component of HyDPPE will comprise hyaluronic acid of a size as is commonly found in natural sources, such as, for example, between about 10,000 to about 5,000,000 Dalton. In some embodiments, the hyaluronic component of HyDPPE will comprise hyaluronic acid of a size between about 10,000 to about 3,000,000 Dalton, or in some embodiments, from about 10,000 to about 1,000,000 Dalton, or in some embodiments, from about 10,000 to about 500,000 Dalton, or in some embodiments, from about 10,000 to about 250,000 Dalton, or in some embodiments, from about 10,000 to about 100,000 Dalton. In some embodiments, the hyaluronic component of HyDPPE will comprise hyaluronic acid of a size between about 10,000 to about 35,000 Daltons.

In some embodiments, optical isomers of HyDPPE, as depicted in Formula I are also to be considered as embodied aspects of this invention.

In some embodiments, HyDPPE is conjugated as a result of the formation of an amide bond between amino head group of phosphatidylethanolamine and the carboxylic group of the hyaluronic acid. The skilled artisan will appreciate the means by which such conjugates may be prepared, including, inter alia, methods as described in U.S. Pat. No. 5,064,817, or in some embodiments, U.S. Pat. No. 7,034,006, or in some embodiments, U.S. Pat. No. 8,865,878 B2; , or in some embodiments, U.S. Pat. No. 8,383,787 B2; herein fully incorporated by reference.

It will be appreciated that the conjugates as described herein may be prepared by any number of means, as known in the art and the invention should not in any way be limited based on the method of producing same.

In some aspects of this invention, as noted, the combination therapy/compositions of this invention will comprise an NSAID, which is a specific inhibitor of the cyclooxygenase-2 enzyme (COX2).

In some aspects, the NSAID envisioned for inclusion in the combination therapy/compositions of this invention and/or for use in accordance with the invention is Celecoxib.

In some aspects, the NSAID envisioned for inclusion in the combination therapy/compositions of this invention and/or for use in accordance with the invention is parecoxib and etoricoxib.

In some aspects, the COX-2 inhibitors envisioned for inclusion in the combination therapy/compositions of this invention and/or for use in accordance with the invention is, for example those mentioned in the following patent applications:

AU9719132, CA2164559, CA2180624, EP-799823, EP-846689, EP-863134, FR2751966, GB2283745, GB2319772, GB2320715, JP08157361, U.S. Pat. Nos. 5,510,368, 5,681,842, 5,686,460, 5,776,967, 5,783,597, 5,824,699, 5,830,911, 5,859,036, 5,869,524, WO9 4 / 1 3635, WO94/20480, WO94/26731, WO95/00501, WO95/21817, WO96/03385, WO96/03387, WO96/06840, WO96/09293, WO96/09304, WO96/13483, WO96/16934, WO96/19462, WO96/19463, WO96/19469, WO96/21667, WO96/23786, WO96/24584, WO96124585, WO96/25405, WO96/26921, WO96/31509, WO96/36617, WO96/36623, WO96/37467, WO96/37469, WO96/38418, WO96/38442, WO96/40143, WO97103953, WO97/09977, WO97/13755, WO97/13767, WO97/14691, WO97/16435, WO97/25045, WO97/25046, WO97125047, WO97/25048, WO97/27181, WO97/28120, WO97/28121, WO97/30030, WO97/34882, WO97/36863, WO97/37984, WO97/38986, WO97/40012, WO97/46524, WO97/46532, WO98/03484, WO98/04527, WO98/06708, WO98/06715, WO98/07425, WO98/11080, WO98/15528, WO98/21195, WO98122442, WO98/28292, WO98/29382, WO98/41511, WO98/41516, WO98/43966, WO98/45294, WO98/46594, WO98/46611, WO98/47890, WO98/51667, WO98/57924, WO99/01455, WO99/05104, WO99/10331, WO99/10332, WO99/11605, WO99/12930, WO99/14194, WO99/14195, WO99/14205, WO99/15505, ZA9704806 and ZA9802828;

In some aspects, the COX-2 inhibitor envisioned for inclusion in the combination therapy/compositions of this invention and/or for use in accordance with the invention is EP-921119, EP-937722, EP-985666, EP-1065204 DE19845446 U.S. Pat. Nos. 5,916,891, 6,083,969, JP11302266, JP2000136182, WO99/18093, WO99/23087, WO99/24404, WO99/25695, WO99/32448, WO99/33796, WO99/35130, WO99/37600, WO99/41224, WO99/43664, WO99/51559, WO99/58523, WO99/61436, WO99/62884, WO99/63939, WO99/64415, WO00/06576, WO00/08024, WO00/10563, WO00/10993, WO00/14082, WO00/17175, WO00/18753, WO00/20371, WO00/20398, WO00/23426, WO00/23433, WO00/26216, WO00/31063, WO00/32567, WO00/39116, WO00/40087, WO00/40243, WO00/50425, WO00/52008, WO00/55139, WO00/61571, WO00/66562, all incorporated herein by reference.

In some aspects, the COX-2 inhibitor envisioned for inclusion in the combination therapy/compositions of this invention and/or for use in accordance with the invention may be provided at a sub-clinical dose and yet still exhibit superior activity in the treatment, etc. of inflammatory and/or allergic conditions, when provided in combination with HyDPPE, as herein described.

In some embodiments, the NSAID will include Celecoxib, Ibuprofen, Vioxx and/or aspirin.

In some embodiments, the NSAID will include derivatives of diarylthiazole, diarylimidazole, mofezolac or derivatives or related forms of same.

According to this aspect and in some embodiments, the NSAID is provided at a dosage that is lower than the typically recommended therapeutic dose, but is provided in combination with HyDPPE, as herein described.

In some embodiments, Celecoxib or Celebrex is provided at a dosage of 100-400 mg/day or less.

In some aspects, the combination therapy/compositions of this invention and/or for use in accordance with the invention, include wherein the NSAID and HyDPPE are administered simultaneously.

In some aspects, the combination therapy/compositions of this invention and/or for use in accordance with the invention, include wherein the NSAID and HyDPPE are administered sequentially.

In some aspects, the combination therapy/compositions of this invention and/or for use in accordance with the invention, include wherein the NSAID and HyDPPE are administered to a subject within 1-72 hours of each other, or any appropriate timing over the duration of the disease and/or condition.

In other embodiments, this invention provides a composition comprising a therapeutically effective amount of an NSAID and a therapeutically effective amount of conjugate of di-palmitoyl (C-16) phosphatidyl ethanolamine and hyaluronic acid (HyDPPE).

It will be understood that the NSAID may be provided in accordance with any embodiment described herein regarding the NSAIDs. Similarly, it will be understood that the Hy- DPPE component of the compositions as described herein may be provided in accordance with any embodiment described herein regarding same.

In other embodiments, this invention provides for use of any composition as described herein, in accordance with any embodiment described herein regarding same for use in treating an inflammatory or allergic disease or condition in a subject.

In some aspects, the combination therapy/compositions of this invention and/or for use in accordance with the invention, are envisioned for use in treating or reducing an inflammatory or allergic disease or condition in a subject.

In some aspects, this invention provides a method of treating, or alleviating symptoms of an inflammatory disease or condition, an allergic disease or condition or a combination thereof, comprising administering to a subject in need thereof a therapeutically effective amount of an NSAID and a therapeutically effective amount of a conjugate of di-palmitoyl (C-16) phosphatidyl ethanolamine and hyaluronic acid (HyDPPE) to a subject in need thereof.

As used herein the phrase “inhibiting” or “treating” refers to reducing, curing, reversing, attenuating, alleviating, minimizing, suppressing or halting the deleterious effects of the indicated disease and/or condition.

In some aspects, when referring to the prevention of a disease herein, such reference is with regard to reduction of incidence of the disease on a population level. In some aspects, such reference may be with regard to a patient suffering from a repeat or relapsing disease, where failure to develop full symptomatology, pathogenesis or severity of the disease as previously occurred in such patient, may serve as an indication of true prevention.

In some aspects, the inflammatory and/or allergic disease and/or condition being treated by the combination therapy/compositions/uses/methods of this invention may include arthritis, including osteoarthritis, asthma, rhinitis, obstructive respiratory disease, colitis, Crohn's disease, central nervous system insult, multiple sclerosis, eczema, contact dermatitis, atopic dermatitis, psoriasis, cardiovascular disease, hemolytic syndromes, sepsis, acute respiratory distress syndrome, pancreatitis, cancer and metastasis, gastric and duodenal ulcer, Covid or any related disease and/or condition.

In some aspects, the inflammatory and/or allergic disease and/or condition being treated by the combination therapy/compositions/uses/methods of this invention may include Sjogren's syndrome or dry eye disease. inflammatory and/or allergic disease and/or condition being treated by the combination therapy/compositions/uses/methods of this invention may include eye diseases and/or conditions, such as conjunctivitis, retinal degeneration, in particular, macular degeneration, and other related disorders and/or conditions.

In some aspects, the inflammatory and/or allergic disease and/or condition being treated by the combination therapy/compositions/uses/methods of this invention may include Crohn's Disease, colitis including ulcerative colitis, immuno-inflammatory intestinal injury, drug-induced enteropathy, ischemia-induced intestinal injury, inflammatory bowel disease, multiple sclerosis, Amyotrophic Lateral Sclerosis (ALS), meningitis, demyelinating diseases of the central and peripheral nervous system, idiopathic demyelinating polyneuropathy or Guillain-Barr syndrome, Alzheimer's disease, Huntington's disease (HD), myasthenia gravis (MG), HIV-associated dementia, fronto-temporal dementia (FTD), stroke, traumatic brain injury, age-related retinal degeneration, encephalomyelitis, chronic inflammatory demyelinating polyneuropathy, cerebral ischemia-induced injury, obstructive respiratory disease, lung injury, intestinal mucosal injury, central nervous system insult, ischemic/reperfusion injury, arterial stenosis and restenosis, multiple sclerosis, sn diseases, contact dermatitis, seboreic dermatitis, psoriasis, conjunctivitis, cardiovascular disease, including prophylaxis for invasive procedures, atherosclerosis, invasive cellular proliferative disorders, primary cancer, metastatic cancer, hemolytic syndromes, sepsis, acute respiratory distress syndrome, tissue transplant rejection syndromes, autoimmune disease, arthritis, or hypersensitivity conjunctivitis. or a combination thereof.

The term “alleviating” as used herein is intended to describe a process by which the severity of a sign or symptom of a disorder is reduced Importantly, the symptoms can be alleviated without eliminating them. In a preferred embodiment, administration of the pharmaceutical composition of the invention leads to elimination of signs or symptoms, but elimination is not necessary. Effective doses are expected to reduce the severity of signs or symptoms.

As used herein, “treating” or “treatment” describes the management and care of a patient for the purpose of combating a disease, condition or disorder, the compounds of the invention, or pharmaceutically acceptable thereof. Includes administration of salts, prodrugs, metabolites, polymorphs or solvates to alleviate the symptoms or complications of a disease, condition or disorder, or to eliminate the disease, condition or disorder.

In one embodiment, “treating” refers to either therapeutic treatment or prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of the subject viral infection, while in one embodiment, “secondary” refers to a symptom that is derived from or consequent to a primary cause. In one embodiment, the compositions and methods for use in the present invention treat primary or secondary symptoms or secondary complications related the pathological condition.

The phrase “therapeutically effective” is intended to qualify the amount of active ingredients used in the treatment of a disease or disorder. This amount will achieve the goal of reducing or eliminating the said disease or disorder.

The pharmaceutical composition of the present invention can be used to treat an indication, i.e., a pathological condition, in a subject in need thereof. The term “subject” as used herein is taken to include humans and other mammals such as cattle, sheep, pigs, goats, dogs, cats, rats, mice, etc., as well as animals including amphibians, birds, reptiles and fish.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment, the combination therapy/compositions/compounds for use in accordance with the methods of this invention may be administered orally, intravenously, intranasally, intraocularly, intramuscularly, subcutaneously or topically, or via any suitable route, including parenteral, intraperitoneal, transderrn.al, rectal, vaginal, buccal, sublingual etc,, with via combined routes of administration envisioned, as well.

Topical formulations composed of the active ingredient of the pharmaceutical composition of the present invention, penetration enhancers, and other biologically active drugs or medicaments may be applied in many ways. A liquid formation can be applied dropwise, from a suitable delivery device, to the appropriate area of skin or diseased skin or mucous membranes and rubbed in by hand or simply allowed to air dry. A suitable gelling agent can be added to the liquid formulation and the preparation can be applied to the appropriate area and rubbed in. For administration to wounds or burns, the active ingredient may be incorporated into dosage forms such as oils, emulsions, and the like. Such preparations may be applied directly to the affected area in the form of :lotions, creams, pastes, ointments, and the :like.

Alternatively, the topical liquid formulation can be placed into a spray device and be delivered as a spray. This type of drug delivery device is particularly well suited for application to large areas of skin affected by dermal pathologies, to highly sensitive skin or to the nasal or oral cavities. Optionally, the pharmaceutical composition may be administered in the form of an ointment or transdermal patch.

The pharmaceutical composition of the present invention may also be administered by other routes which optimize uptake by the mucosa, e.g., vaginal (especially in the case of treating vaginal pathologies), rectal and intranasal routes of administration. Furthermore, the pharmaceutical composition may be adapted for delivery through mucosal tissue or epithelia. If administered intranasally, the pharmaceutical composition will typically be administered in an aerosol form, or in the form of drops. This may be especially useful for treating lung pathologies.

Suitable formulations can be found in A. Gennaro (2000) “Remington: The Science and Practice of Pharmacy”, 20th edition, Lippincott, Williams, & Wilkins Pharmaceutical Dosage Forms and Drug e:livery Systems (1999) H. C. Ansel et al., eds 7^{th} ed., Lippincott, Williams, & Wilkins and Handbook of Pharmaceutical Excipients (2000) A. FL Kibbe et al., eds., 3rd ed. Amer. Pharmaceutical Assoc. each of which is incorporated herein by reference.

Depending on the intended mode of administration, the composition used may be in the form of solid, semi-solid or liquid dosage forms, such as for example, tablets, suppositories, pills, capsules, powders, liquids, suspensions, or the like, preferably in unit dosage forms suitable for single administration of precise dosages. The pharmaceutical composition of the present invention and a pharmaceutically acceptable diluent, carrier, excipient, adjuvant, or auxiliary agent. it is preferred that the pharmaceutically acceptable carrier be one which is chemically inert to the active therapeutic protein and which has no detrimental side effects or toxicity under the conditions of use. The choice of carrier is determined partly by the particular active ingredient, as well as by the particular method used to administer the composition. Accordingly, there are a wide variety of suitable formulations of the pharmaceutical compositions of the present invention.

Suitable excipients are, in particular, fillers such as saccharides (e.g., lactose or sucrose, mannitol, sorbitol, etc.) cellulose preparations and/or calcium phosphates (e.g., tricalcium phosphate, calcium hydrogen phosphate, etc.) as well as binders such as starch paste using, for example, maize starch, wheat starch, rice starch, potato starch, gelatin, tragacanth, methylcellulose, hydroxypropylmethylcellulose, sodium carboxymethylcellulose, and/or polyvinyl pyrrolidine.

Injectable formulations for parenteral administration can be prepared as liquid suspensions, solid forms suitable for solution or suspension in liquid prior to injection, or as emulsions. Suitable excipients are, for example, water, saline, dextrose, glycerol, ethanol or the like. In addition. if desired, the pharmaceutical composition to be administered may also contain minor amounts of non-toxic auxiliary agents such as wetting or emulsifying agents, pH buffering agents and the like, such as for example, sodium acetate, sorbitan monolaurate, triethanolamine oleate, etc.

Aqueous injection suspensions may also contain substances that increase the viscosity of the suspension, including, for example, sodium carboxymethylcellulose, sorbitol, and/or dextran. Optionally, the suspension may also contain stabilizers.

The parenteral formulations can be present in unit dose or multiple dose sealed containers, such as ampules and vials, and can be stored in a freeze-dried (lyophilized) condition requiring only the addition of the sterile liquid carrier, e,g., water, for injections immediately prior to use. Extemporaneous injection suspensions can be prepared from sterile powders, granules, and tablets of the kind previously described.

For oral administration, a pharmaceutically acceptable, non-toxic composition is formed by the incorporation of any of the normally employed excipients, such as, for example, mannitol, lactose, starch, magnesium stearate, sodium saccharine, talcum, cellulose, sodium crosscarmellose, glucose, gelatin, sucrose, magnesium carbonate, and the like. Such compositions include suspensions, tablets, dispersible tablets, pills, capsules, powders, sustained release formulations and the like. Formulations suitable for oral administration can consists of liquid suspensions such as effective amounts of the drug encapsulating gagomer particles suspended in diluents such as water, saline, or orange juice; sachets, lozenges, and troches, each containing a predetermined amount of the active ingredient as solids or granules; powders, suspensions in an appropriate liquid; and suitable emulsions. Liquid formulations may include diluents such as water and alcohols, e.g,, ethanol, benzyl alcohol, and the polyethylene alcohols, either with or without the addition of a pharmaceutically acceptable surfactant, suspending agents, or emulsifying agents.

When the composition is a pill or tablet, it will contain, along with the active ingredient, a diluent such as lactose, sucrose, dicalcium phosphate, or the like; a lubricant such as magnesium stearate or the like; and a binder such as starch, gum acacia, gelatin, polyvinylpyrrolidine, cellulose and derivatives thereof, and the like.

Tablet forms can include one or more of lactose, sucrose, mannitol, corn starch, potato starch, alginic acid, microcrystalline cellulose, acacia, gelatin, guar gum, colloidal silicon dioxide, crosscarmellose sodium, talc, magnesium stearate, calcium stearate, zinc stearate, stearic acid, preservatives. flavoring agents, pharmaceutically acceptable disintegrating agents, moistening agents, and pharmacologically compatible carriers.

Capsule forms can be of the ordinary hard- or soft-shelled gelatin type containing, for example, surfactants, lubricant, and inert fillers, such as lactose, sucrose, calcium phosphate, and corn starch.

Lozenge forms can contain the drug encapsulating gagomer particles in a carrier, usually sucrose and acacia or tragacanth, as well as pastilles comprising the active ingredient in an inert base such as gelatin or glycerin, or sucrose and acacia.

The amount of the active ingredient in the pharmaceutical composition of the present invention to be administered to any given patient must be determined empirically, and will differ depending upon the condition of the patients. Relatively small amounts of the pharmaceutical composition can be administered at first, with steadily increasing dosages if no adverse effects are noted. Of course, the maximum safe toxicity dosage as determined in routine animal toxicity tests should never be exceeded.

Pharmaceutical compositions within the scope of the present invention include all compositions wherein the active ingredients are contained in an amount effective to achieve their intended purpose. While individual needs vary, determination of optimal ranges of effective amounts of each compound is within the skill of the art. The dosage administered will depend upon the age, health, and weight of the individual recipient thereof as well as upon the nature of any concurrent treatment and the effect desired.

As used herein the term “about” refers to +/−10% variance from the stated value.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Effects of Cox-2 Inhibitors And HyDDPE on LTB4 Production by the Hippocampus (HC) and Hypothalamus (HT) Of Animals Stimulated with LPS

### Materials and Reagents

Di-palmitoyl (C-16) phosphatidyl ethanolamine (DPPE) conjugated to hyaluronic acid (Hy) was prepared using methods previously established (see for example, U.S. Pat. Nos. 5064817 and 7,034,006, herein fully incorporated by reference. The molecular weight of the hyaluronic acid in the conjugate was from 10,000-30,000 Da. Celecoxib was purchased Glentham Life Scinces (# GP8233), and LPS was purchased from Sigma Aldrich ((# L3129).

### Experimental protocol

Male Sprague-Dawley rats were purchased from Harlan laboratories, fed ad libitum and housed in accordance with animal facility guidelines. Animals were assigned 10 each to the 7 groups, in accordance with the treatment protocol described below. Body temperature (BT) was measured at all treatment time-points, to evaluate the effects of drug treatment on the LPS-induced changes in BT. On the second day of the experiment, 2 hours post- drug treatment, rats were sacrificed by decapitation after a short anesthesia (with a mixture of isoflurane-oxygen in air inhalation), blood was collected and brain regions (hypothalamus and hippocampus) immediately extracted. Brain regions were manually homogenized in a PBS solution containing a cocktail of phosphatase/protease inhibitors. Homogenates were centrifuged at 4° C., 10,000 rpm, for 10 minutes. Supernatants were collected and stored at −80° C. for further determination.

Leukotriene B4 (LTB4) levels were determined in the hypothalamus and hippocampus samples, using commercially available ELISA kits.

### Treatment Groups

Seven groups of rats, ten rats in each group, were administered treatment intra-peritoneally, according to the following treatment regimens:
1)- Control group- Animals were treated with vehicle control (NaCl 0.9% 0.2 ml);
2) LPS: Animals were treated with vehicle control at 6 and 2 hours prior to LPS (1 mg/kg) injection. 22 hours following the LPS injection (2 hours before sacrifice), animals were administered a further vehicle control injection, then sacrificed.

3) Celecoxib: animals were treated with Celecoxib 10 mg/kg (26 μmole/kg) in saline, at a timing to match 6 and 2 hours before LPS injection of the LPS group, and subsequently administered Celecoxib 20 mg/kg at 2 hours before sacrifice on the following day. These animals were not given any LPS however, and instead were provided vehicle control at the time of LPS injection.

4) HyDPPE: animals were treated with 50 mg/kg (5 μmole/kg) in saline, at 6 and 2 hours before LPS injection of the LPS group, and subsequently administered HyDPPE at 50 mg/kg the following day, 2 hours before sacrifice. These animals were not given any LPS however, and instead were provided vehicle control at the time of LPS injection.

5) LPS +Celecoxib: animals were treated with Celecoxib 10 mg/kg (26 μmole/kg) in saline, at a timing to match 6 and 2 hours before LPS (1 mg/kg) injection and subsequently administered Celecoxib 20 mg/kg at 2 hours before sacrifice on the following day.

6) LPS +HyDPPE: animals were treated with HyDPPE 50 mg/kg (5 μmole/kg) in saline, at 6 and 2 hours before LPS (1 mg/kg) injection and subsequently administered HyDPPE at 50 mg/kg the following day, 2 hours before sacrifice.

7) LPS +Celecoxib +MFAIDs: animals were treated with Celecoxib 10 mg/kg (26 μmole/kg) in saline, and HyDPPE 50 mg/kg (5 μmole/kg) in saline, at 6 and 2 hours before LPS (1 mg/kg) injection and subsequently administered Celecoxib 20 mg/kg and HyDPPE at 50 mg/kg mg/kg the following day, 2 hours before sacrifice.

Table 1 plots the results of LTB4 production in each group, in the Hippocampus samples.

Table 1 plots the results of LTB4 production in each group, in the Hippocampus samples.

| | | | | | | | LPS + |
|---|---|---|---|---|---|---|---|
| | | Celecoxib | HyDPPE | | LPS + | LPS + | Celecoxib + |
| | Control | Alone | Alone | LPS | Celecoxib | HyDPPE | HyDPPE |
| MEAN | 25.77 | 44.82 | 24.04 | 53.34 | 66.63 | 37.94 | 22.97 |
| SD | 12.38 | 11.11 | 5.69 | 13.96 | 11.84 | 17.01 | 13.63 |
| SEM | 3.91 | 3.51 | 1.80 | 4.42 | 3.74 | 5.38 | 4.31 |

FIG. 1 plots these results, in graphic form. As clearly evident, in Hippocampus samples,

LTB4 production vs. Control increased due to LPS (P <0.0001, Student's T test) or Celecoxib (P<0.0001, Student's T test) administration, which was even more pronounced when the two were administered in combination (P<0.0001, Student's T test). In marked contrast, administration of HyDPPE counteracted the LPS-induced production of LTB4 (P<0.0001, Student's T test), including even in the face of the combination of LPS and Celecoxib (P<0.00001, Student's T test).

Hypothalamus samples were evaluated as well, and the results are provided in Table 2:

| | | | | | | | LPS + |
|---|---|---|---|---|---|---|---|
| | | | | | LPS + | LPS + | Celecoxib + |
| | Control | Celecoxib | HyDPPE | LPS | Celecoxib | HyDPPE | HyDPPE |
| MEAN | 23.40 | 43.88 | 29.58 | 53.83 | 57.55 | 37.33 | 43.19 |
| SD | 9.20 | 18.85 | 6.60 | 11.69 | 18.74 | 12.23 | 23.00 |
| SEM | 2.91 | 5.96 | 2.09 | 3.70 | 5.93 | 3.87 | 7.27 |

FIG. 2 plots these results, in graphic form. Here, as well, in the hypothalamus

samples it is evident that vs. Control, LTB4 production increased due to LPS (P<0.0001, Student's T test) or Celecoxib (P<0.05, Student's T test) administration. In marked contrast, administration of HyDPPE counteracted the LPS-induced production of LTB4 (P<0.005, Student's T test).

It will be understood that various alternatives and modifications may be devised by those skilled in the art. However, these should not be viewed as limitations upon the practice of these teachings, as those skilled in the art, when guided by the foregoing teachings, may derive other suitable characteristics of a similar or different nature. The present invention is intended to embrace all such alternatives, modifications and variances that fall within the scope of the appended claims.

I claim:

1. A combination therapy for treating an inflammatory or allergic disease or condition, said combination therapy comprising a therapeutically effective amount of a non-steroidal anti- inflammatory drug (NSAID) and a therapeutically effective amount of conjugate of di-palmitoyl (C- 16) phosphatidyl ethanolamine and hyaluronic acid (HyDPPE).

2. The combination therapy of claim 1, wherein said NSAID comprises a cyclooxygenase inhibitor.

3. The combination therapy of claim 1, wherein said NSAID is a specific inhibitor of the cyclooxygenase-2 enzyme (COX2).

4. The combination therapy of claim 1, wherein NSAID is Celecoxib.

5. The combination therapy of claim 1, wherein said NSAID is Vioxx.

6. The combination therapy of claim 1, wherein said NSAID is aspirin.

7. The combination therapy of any one of claims 2-6, wherein said NSAID is provided at a sub- clinical dosage.

8. The combination therapy of claim 1, wherein said HyDPPE comprises hyaluronic acid of between 10-30 kDA in size.

9. The combination therapy of claim 1, wherein said NSAID and said HyDPPE are administered simultaneously.

10. The combination therapy of claim 1, wherein said NSAID and said HyDPPE are administered sequentially.

11. The combination therapy of claim 1, wherein each of said NSAID and said HyDPPE are administered to a subject within 6 hours of each other.

12. The combination therapy of claim 1, wherein said inflammatory or allergic disease or condition is asthma, rhinitis, obstructive respiratory disease, colitis, Crohn's disease, central nervous system insult, multiple sclerosis, arthritis, osteoarthritis, contact dermatitis, psoriasis, eczema, atopic dermatitis, cardiovascular disease, hemolytic syndromes, sepsis, acute respiratory distress syndrome, cancer and metastasis, pancreatitis, gastric and duodenal ulcer or Covid.

13. The combination therapy of claim 1, wherein said inflammatory or allergic disease or condition is an eye disease and/or disorder.

14. A composition comprising a therapeutically effective amount of an NSAID and a therapeutically effective amount of conjugate of di-palmitoyl (C-16) phosphatidyl ethanolamine and hyaluronic acid (HyDPPE).

15. The composition of claim 14, wherein said NSAID comprises a cyclooxygenase inhibitor.

16. The composition of claim 14, wherein said NSAID is a specific inhibitor of the cyclooxygenase-2 enzyme (COX2).

17. The composition of claim 14, wherein NSAID is Celecoxib.

18. The composition of claim 14, wherein said NSAID is Vioxx.

19. The composition of claim 14, wherein said NSAID is aspirin.

20. The composition of any one of claims 15-19, wherein said NSAID is provided at a sub-clinical dosage.

21. The composition of claim 14, wherein said HyDPPE comprises Hyaluronic acid of MW between 10-30 kDA in size.

22. A Composition comprising a therapeutically effective amount of an NSAID and a therapeutically effective amount of conjugate of di-palmitoyl (C-16) phosphatidyl ethanolamine and hyaluronic acid (HyDPPE) for use in treating an inflammatory or allergic disease or condition in a subject.

23. The composition for use of claim 22, wherein said NSAID comprises a cyclooxygenase inhibitor.

24. The composition for use of claim 22, wherein said NSAID is a specific inhibitor of the cyclooxygenase-2 enzyme (COX2).

25. The composition for use of claim 22, wherein NSAID is Celecoxib.

26. The composition for use of claim 22, wherein said NSAID is Vioxx.

27. The composition for use of claim 22, wherein said NSAID is aspirin.

28. The composition for use of any one of claims 22-26, wherein said NSAID is provided at a sub-clinical dosage.

29. The composition for use of claim 22, wherein said HyDPPE comprises hyaluronic acid of between 10-30 kDA in size.

30. A method of treating or alleviating symptoms of an inflammatory disease or condition, an allergic disease or condition or a combination thereof, said method comprising administering to a subject in need thereof a therapeutically effective amount of an NSAID and a therapeutically effective amount of a conjugate of di-palmitoyl (C-16) phosphatidyl ethanolamine and hyaluronic acid (HyDPPE) to a subject in need thereof.

31. The method of claim 30, wherein said NSAID comprises a cyclooxygenase inhibitor.

32. The method of claim 30, wherein said NSAID is a specific inhibitor of the cyclooxygenase-2 enzyme (COX2).

33. The method of claim 30, wherein NSAID is Celecoxib.

34. The method of claim 30, wherein said NSAID is Vioxx.

35. The method of claim 30, wherein said NSAID is aspirin.

36. The method of any one of claims 30-34, wherein said NSAID is provided at a sub-clinical dosage.

37. The method of claim 30, wherein said HyDPPE comprises hyaluronic acid of between 10-30 kDA in size.

38. The method of claim 30, wherein said NSAID and said HyDPPE are administered to said subject simultaneously.

39. The method of claim 30, wherein said NSAID and said HyDPPE are administered to said subject sequentially.

40. The method of claim 30, wherein said inflammatory or allergic disease or condition is asthma, rhinitis, obstructive respiratory disease, colitis, Crohn's disease, central nervous system insult, multiple sclerosis, arthritis, osteoarthritis, contact dermatitis, psoriasis, eczema, atopic dermatitis, cardiovascular disease, hemolytic syndromes, sepsis, acute respiratory distress syndrome, cancer and metastasis, pancreatitis, gastric and duodenal ulcer or Covid.

41. The method of claim 30, wherein said inflammatory or allergic disease or condition is an eye disease or disorder.
